(19) United States
(12) Patent Application Publication
(10) Pub. No.: US 2007/0260126 A1
(43) Pub. Date: Nov. 8, 2007

(54) Medical information acquisition and display system

(76) Inventors: Michael Haumann, Oberhausen (DE); Hugo Geuvens, Boutersem (BE)

Correspondence Address: Siemens Corporation;Intellectual Property Department, 170 Wood Avenue South, Iselin, NJ, 08830, US

(21) Appl. No.: 11/402,719

(22) Filed: Apr. 12, 2006

(51) Int. Cl. A61B 5/00

(52) U.S. Cl. 600/300

(57) ABSTRACT

A user interface system for presenting medical information related to a patient episode includes an acquisition processor for acquiring data related to a patient episode in response to received data identifying the particular episode. The patient episode related data includes data from a time period during the episode, and from at least one of: data from a time period before the episode, and data from a time period after the episode. A display processor provides data representing a single display image presenting a timeline together with user selectable items of surgical operation related data acquired by the acquisition processor for a patient during a user selected time period. A command processor initiates presentation of the single display image in response to a user command.

### FIELD OF THE INVENTION

The present invention relates to a system for acquiring and displaying medical information, and in particular to a user interface system for presenting medical information supporting a patient surgical procedure and related treatment.

### BACKGROUND OF THE INVENTION

Healthcare providers, such as doctors, hospitals, clinics etc., maintain patient medical data in a computerized database. Some databases contain general clinical medical data and are termed a clinical information systems (CIS). Other databases contain patient medical data more specifically related to surgical procedures and are termed operating room management systems (ORMS). The data stored in such a database forms at least a part of a patient medical record. Computerized database systems (CIS or ORMS) permit portions of the patient medical data to be easily selected by a user, such as a doctor or nurse, and displayed for that user. Typically, data is retrieved from the database in a manner related to patient visits to the healthcare provider. That is, a user selects one of the patient visits, and data related to that visit (e.g. patient interview results, examination results, lab test results, X-ray and other test results, diagnoses, and proposed treatments) is displayed for the user to review.

Computerized patient medical records may contain a large amount of data; much more information than is needed at one time to make decisions in the treatment of the patient. In existing systems users of a CIS and/or ORMS, may also either search for relevant clinical information or may select data within a certain period of time within the overall Medical Record. The result of these searches often contains too little or too much clinical data and fails to find and identify to a user the required pertinent information. A system according to invention principles addresses these deficiencies and related problems.

### BRIEF SUMMARY OF THE INVENTION

In accordance with principles of the present invention, a user interface system for presenting medical information related to a patient episode includes an acquisition processor for acquiring data related to a patient episode in response to received data identifying the particular episode. The patient episode related data includes data from a time period during the episode, and from at least one of: data from a time period before the episode, and data from a time period after the episode. A display processor provides data representing a single display image presenting a timeline together with user selectable items of surgical operation related data acquired by the acquisition processor for a patient during a user selected time period. A command processor initiates presentation of the single display image in response to a user command.

### BRIEF DESCRIPTION OF THE DRAWING

In the drawing:

FIG. 1 is a block diagram of a user interface system according to principles of the present invention;

FIG. 2 is a timeline diagram illustrating a patient medical record and various views of that record; and

FIG. 3 and FIG. 4 illustrate display images which are a portion of a user interface according to principles of the present invention.

### DETAILED DESCRIPTION OF THE INVENTION

A processor, as used herein, operates under the control of an executable application to (a) receive information from an input information device, (b) process the information by manipulating, analyzing, modifying, converting and/or transmitting the information, and/or (c) route the information to an output information device. A processor may use, or comprise the capabilities of, a controller or microprocessor, for example. The processor may operate with a display processor or generator. A display processor or generator is a known element for generating signals representing display images or portions thereof. A processor and a display processor comprises any combination of, hardware, firmware, and/or software.

An executable application, as used herein, comprises code or machine readable instructions for conditioning the processor to implement predetermined functions, such as those of an operating system, medical information acquisition and display system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters.

A user interface (UI), as used herein, comprises one or more display images, generated by the display processor under the control of the processor. The UI also includes an executable procedure or executable application. The executable procedure or executable application conditions the display processor to generate signals representing the UI display images. These signals are supplied to a display device which displays the image for viewing by the user. The executable procedure or executable application further receives signals from user input devices, such as a keyboard, mouse, light pen, touch screen or any other means allowing a user to provide data to the processor. The processor, under control of the executable procedure or executable application manipulates the UI display images in response to the signals received from the input devices. In this way, the user interacts with the display image using the input devices, enabling user interaction with the processor or other device.

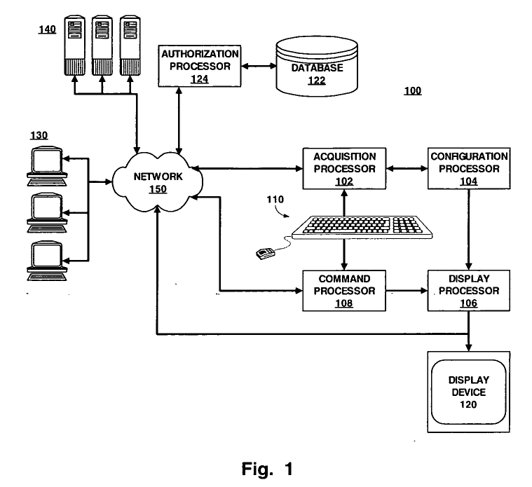

FIG. 1 is a block diagram of a user interface system 100 for presenting medical information related to a patient episode, according to principles of the present invention. A patient episode is a health related occurrence which generally has a time limited duration. That is, it has a definable beginning and ending time. It may be a medical illness or condition, or a treatment, such as a surgical procedure.

In the following description, a patient episode is specifically a surgical procedure. In FIG. 1, the user interface system 100 includes an acquisition processor 102. The acquisition processor 102 is bidirectionally coupled to a configuration processor 104 and to a network 150. An output terminal of the configuration processor 104 is coupled to a first input terminal of a display processor 106. An output terminal of the display processor 106 is coupled to an input terminal of a display device 120 and to the network 150. The user interface system 100 further comprises a command processor 108 bidirectionally coupled to the network 150 and having an output terminal coupled to a second input terminal of the display processor 106. Input devices 110 are coupled to respective input terminals of the command processor 108 and the acquisition processor 102. In FIG. 1, the input devices 110 are illustrated as a keyboard and mouse, though any similar device capable of communicating with the acquisition processor 102 and/or the command processor 108 may be used. For example, the input devices 110 may also include (a) devices monitoring patient physiological parameters (e.g. vitals signs, etc.), and/or (b) patient treatment devices supplying patient physiological data and data related to the treatment being administered (e.g. infusion pumps, anesthesiology equipment, ventilators). The user interface system 100 further includes a database 122 which is bidirectionally coupled to the network 150 through an authorization processor 124. The network 150 is coupled to a plurality of other nodes, such as workstations 130 and servers 140. The servers 140 may store data from other healthcare related data systems, such as: (a) laboratory, (b) other clinical information systems for providing test results, and (c) other medical information.

In operation, the network 150 enables the nodes to which it is connected to intercommunicate. The acquisition processor 102 acquires data related to a patient episode, specifically a surgical operation, in response to receiving data identifying that particular surgical operation. More specifically, in the illustrated embodiment, the data identifying the particular surgical operation contains data identifying a particular set of orders for treatments and related services initiated for processing by a physician and associated with the particular surgical operation. The acquisition processor 102 may automatically acquire the surgical operation related data in response to a physician initiating processing of a particular set of orders via a treatment order entry system. Such a treatment order entry system is not explicitly shown in FIG. 1, but may be implemented on a server 140 and controlled by a user at a workstation 130. As described above, the data identifying the particular surgical operation contains data identifying a particular set of orders for treatments and related services associated with the operation. The treatment order entry system receives orders from a physician, schedules healthcare facility resources, such as an operating room, schedules a date and time for the operation, and supplies the identification data to the acquisition processor 102. The display processor 106 terminates processing of the display image in response to cancellation of the set of orders, as received e.g. from the treatment order entry system.

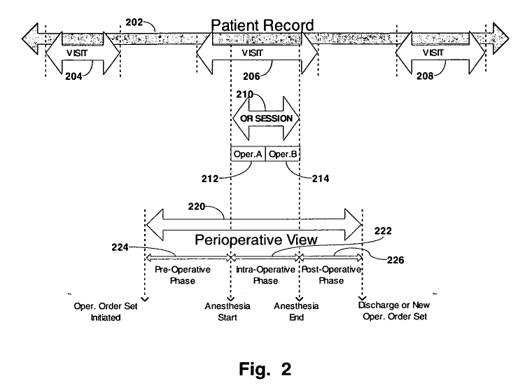

FIG. 2 is a timeline diagram illustrating a patient medical record and various views of that record. In FIG. 2, time is illustrated on the horizontal axis. A patient record is represented by a double ended line 202 across the top. This is intended to represent that patient related medical data exists before and after the period of time illustrated in FIG. 2. As described above, existing CIS systems provide views of the data in the patient medical record 202 related to visits by the patient to the healthcare provided. This is illustrated in FIG. 2 by the arrows 204, 206 and 208, each indicating a period of time of a respective visit of the patient to the healthcare provider. In existing systems, a user interface (UI) is presented to a user via the display device 120 (FIG. 1) which gives access to the patient related medical data located in the patient medical record 202 during those visit time periods 204, 206, 208. The user (if appropriately authorized) is able to select, view, edit, enter, and otherwise interact with the available patient related medical data displayed in the UI via the input devices 110.

The data retrieved by the acquisition processor 102 (FIG. 1) according to principles of the present invention is related, not to visits, but to a patient episode. The patient episode includes data from a time period during the episode, and at least one of data from a time period before and a time period after the episode. When the episode is specifically a surgical procedure, the patient surgical operation related data includes intra-operative data and at least one of: pre-operative data, and post-operative data associated with the particular surgical operation for a particular patient.

This is illustrated in more detail in FIG. 2. During the visit 206, the patient is scheduled for an operating room (OR) session 210. The OR session 210 will include two operations: operation A 212 and operation B 214. The time period when these operations will take place, which begins when anesthesia is administered to the patient and ends when the anesthesia ends, is termed the intra-operative phase 222. Patient medical data gathered during this time period is termed intra-operative data. However, patient medical data relevant to the operation may be gathered in the patient medical record 202 at times before and/or after the intra-operative phase 222, including times before and/or after the patient visit 206. The time period before the intra-operative phase 222 is termed pre-operative phase 224 and patient medical data gathered during this phase is termed pre-operative data. The time period after the intra-operative phase 222 is termed the post-operative phase 226 and patient medical data gathered during this phase is termed post-operative data. The period of time including the pre-operative phase 224, intra-operative phase 222 and post operative phase 226 is termed the perioperative view 220.

Typically, or by default, the pre-operative phase 224 begins when the set of orders for treatments and related services is initiated for processing by a physician; and the post-operative phase 226 ends when the patient is discharged or another set of orders for treatments and related services is initiated. However, default start and end times of the perioperative view may be changed by the administration of the healthcare facility for the users. In addition, different default starting and ending times may be set for different classes of users (e.g. doctors, nurses, OR scheduling administration personnel, etc.). An individual user may set starting and ending times as desired to encompass the time period of interest to that user. These individual user settings may be remembered from one session to another, or may reset to the user preferred times when a new session is started. It is also possible that a user's settings for a particular patient may be saved from session to session, so that at the next session related to that patient, the previously set starting and ending times are used.

As described above, the acquisition processor 102 acquires surgical operation related data in response to receiving data identifying a particular surgical operation. The acquisition processor 102 may bidirectionally communicate with a plurality of different information sources to acquire this surgical operation related data. Typically, the database 122 provides medical data for a patient to the acquisition processor 102. However, such medical data may also be provided by other databases (not shown), such as pharmacy and/or laboratory databases. In addition such medical data may be provided by users at workstations 130, or may be retrieved by the acquisition processor 102 from servers 140. A user may also provide data to the acquisition processor 102 via the input devices 110.

The command processor 108 initiates presentation of a single display image in response to a user command requesting such a display. The user may provide such a command using the input devices 110 or may access the command processor 108 via the network 150 using a workstation 130. In response to the initiation by the command processor 108, the display processor 106 generates data representing a single display image functioning as the display portion of a user interface (UI). The display device 120 receives the image representative data and displays the UI display image represented by that data. The display image representative data may also be supplied to a workstation 130 via the network 150. The display device at the workstation 130 displays the UI image and the input devices at the workstation 130 permit the user to interact with that image in a manner described in more detail below.

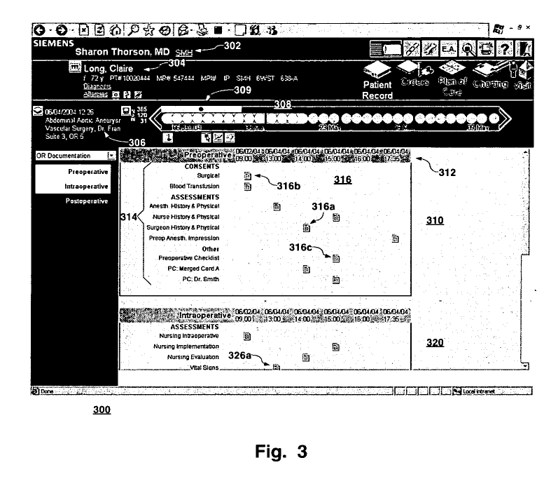

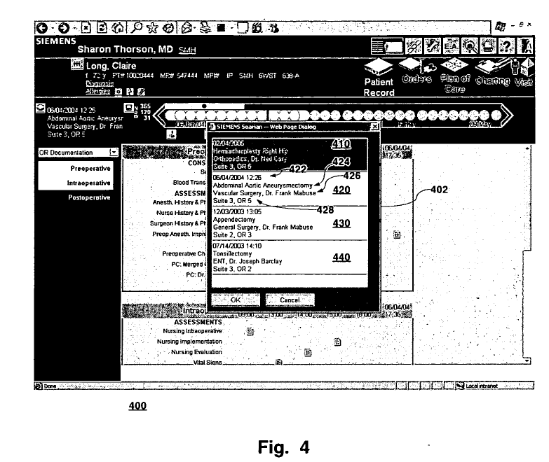

FIG. 3 and FIG. 4 illustrate display images which are a portion of a UI according to principles of the present invention. In FIG. 3, the UI display image 300 is in the form of a timeline representing a user selected time period, together with user selected items of data related to the surgical operation acquired by the acquisition processor 102. User identification information 302 is displayed at the top of the image. Patient identification information 304 is listed below that, along with other pertinent data related to the patient, including medical record number, hospital, room number, etc. Below that, a first data display section 310 presenting pre-operative patient surgical operation related data is displayed. Dates and times 312 are displayed across the top of the data display section 310. Classes of data 314 are listed at the left hand side of the data display section 310: specifically, Consents, Assessments and Other data are listed on FIG. 3. In area 316, display elements representing surgical operation related data are displayed. A portion of a second data display section 320 presenting intra-operative patient surgical operation related data is displayed. The second data display section 320 is arranged similarly to the first data display section 310. A third data display section (not shown), presenting post-operative patient surgical operation related data, may also be displayed for a user.

Referring now to FIG. 4, because data related to more than one surgical procedure for the patient may be available in the patient record 202 (FIG. 2), the display processor 106 (FIG. 1) may further generate data representing a UI display 400 which includes an image window 402 enabling a user to select a particular surgical operation for the particular patient from data representing a plurality of different operations performed on that patient. The single display image 400, and in particular the selection image window 402, includes entries 410, 420, 430, 440 which respectively identify, two or more of: (a) the date and time of surgery 422, (b) the type of surgery performed 424, (c) the participating surgeons and physicians 426, and (d) the place of surgery 428, for the plurality of different operations. The user interacts with the system 100, e.g. via the display device 120 and the input devices 110 or via a workstation 130 and network 150, to select one of the surgeries listed in the selection image window 402.

Referring back to FIG. 3, in response to selection of one of the surgeries displayed in the selection image window 402 (FIG. 4) (for example, the surgery represented by the entry 420), the display processor 106 (FIG. 1) generates data conditioning the display device 120 to display the surgery identification data 306. Concurrently, the acquisition processor 102 (FIG. 1) acquires surgical operation related data for the selected surgical procedure. The surgical operation related data is displayed in the display area 316.

As described above, the display processor 106 generates data representing the single display image 300 in the form of a time line. While the staring and ending times of the timeline may be selected by a user, the timeline is associated with a patient surgical procedure and related treatment, and includes at least the day of surgery or scheduled day of surgery. In FIG. 3, a day selector UI image element 308 is displayed which allows a user to easily select a viewing period. Dates are displayed on the bottom of the day selector. The single display image includes display attributes for (a) highlighting the day of surgery, and/or (b) highlighting the scheduled day of surgery. More specifically, in the illustrated embodiment, the day that the selected surgery was performed is highlighted by a bar of contrasting color, and including a large dot 309. The same bar is displayed to highlight a day for which a future operation is scheduled.

The surgical operation related data may include clinical and non-clinical data related to the particular patient. In the illustrated embodiment, access to the data is provided by representing the respective data items as icons displayed at appropriate locations on the displayed timeline; for example, (a) medical assessment information of the particular-patient provided by a physician 316a, (b) a consent form associated with treatment of the particular patient 316b, and/or (c) a questionnaire associated with treatment of the particular patient 316c.

Referring again to FIG. 1, the user interacts with the acquisition processor 102 by manual data entry, i.e. by using the input devices 110 and/or a workstation 130 via network 150 to select a desired item of data related to the surgical operation by e.g. ‘clicking on’ the displayed icon representing the desired data item. For example, if an administrator wishes to review the patient surgical consent form, the administrator would operate the input devices 110 and/or workstation 130 to ‘click on’ the icon 316b (FIG. 3) representing the patient surgical consent form. In response, the acquisition processor 102 acquires the surgical operation related data represented by the selected data item and conditions the display processor 106 to display the selected data for the user. If, for example, the user selected item is the patient surgical consent form, then the form would be displayed on the display device 120 and/or workstation 130 in a form readable by the user. The user selected items of surgical operation related data may also comprise patient clinical medical parameters, which are displayed in an appropriate manner. That is, a user may select vital signs 326a, which may include ECG and blood pressure medical data, among other things. If ECG data is selected, data representing an image of the selected ECG leads in waveform format is generated by the display processor 106 and displayed by the display device 120 and/or workstation 130. Similarly, if blood pressure data is selected, the diastolic and systolic pressures are displayed in numeric format, and so forth.

As described above, the starting and ending times of the perioperative time line 220 (FIG. 2) may be selected by the user. In addition, some patient related data (e.g. consents, assessments) may be specified by a user to appear on the time line UI display image 300, and other data (e.g. vital signs, laboratory results) may be specified by the user to be excluded from the time line UI display image 300. This permits the surgical procedure data which is most relevant to the user to be displayed, and relieves the user from searching for relevant data among less relevant, or irrelevant data, ensuring that relevant data will not be missed because it is lost in the large amount of available data. The surgical procedure representative data included in the display image may also be specified by administrative or supervisory personnel at the healthcare facility for different classes of users (e.g. doctors, nurses, administrative personnel, etc.), or may be specified by the supplier of the system 100. The configuration processor 104 (FIG. 1) cooperates with the acquisition processor 102 to supply the surgical related data which has been specified by the user to the display processor 106 for display in the UI display image 300 by the display device 120 and/or workstation 130. This enables a user to select which information is presented in the UI display image 300 (FIG. 3) including the user selected perioperative time period.

It is further required that no one but authorized people may access medical data. As described above, medical data is typically stored in the database 122. The authorization processor 124 inhibits the display processor 106 from providing data representing the single display image in response to a determination, based on received user identification information, that a user is not authorized to access the surgical operation related data of the particular patient. This is done in the authorization processor 124 by maintaining, for the respective patients, a list of people authorized to access the medical information for that patient. The authorization processor 124 requires identification information from the user requesting medical data about a patient, and retrieves and returns such information to those who are on the list of authorized people for that patient.

What is claimed:

1. A user interface system for presenting medical information related to a patient episode, comprising:
an acquisition processor for acquiring data related to a patient episode, including data from a time period during the episode, and from at least one of: data from a time period before the episode, and data from a time period after the episode, in response to received data identifying the particular episode;
a display processor for providing data representing a single display image in the form of a timeline representing a user defined time period, together with user selectable items of data related to the patient episode acquired by the acquisition processor; and
a command processor for initiating presentation of the single display image in response to a user command.

2. The system of claim 1 wherein the patient episode is a surgical procedure.

3. A user interface system for presenting medical information supporting a patient surgical procedure and related treatment, comprising:
an acquisition processor for acquiring surgical operation related data including intra-operative data and at least one of, pre-operative data, and post-operative data associated with a particular surgical operation for a particular patient in response to received data identifying said particular surgical operation;
a display processor for providing data representing a single display image in the form of a timeline representing a user selected time period, together with user selectable items of data related to said surgical operation acquired by said acquisition processor; and
a command processor for initiating presentation of said single display image in response to user command.

4. A system according to claim 3, further comprising at least one input device coupled to the acquisition processor, wherein the input device communicates with the acquisition processor when the acquisition processor is acquiring said surgical operation related data.

5. A system according to claim 4, wherein the input device comprises a medical monitoring device providing data representing patient physiological parameters, coupled to the acquisition processor, wherein the acquisition processor accesses the medical monitoring device when acquiring said surgical operation related data.

6. A system according to claim 4, wherein the input device comprises a medical treatment device, providing data representing at least one of: (a) patient physiological parameters, and (b) patient treatment data, coupled to the acquisition processor, wherein the acquisition processor accesses the medical treatment device when acquiring said surgical operation related data.

7. A system according to claim 3, further comprising a server, coupled to the acquisition processor via a network, containing surgical operation related data, coupled to the acquisition processor via a network, wherein the acquisition processor accesses the server when acquiring said surgical operation related data.

8. A system according to claim 3, further comprising a workstation, coupled to the acquisition processor via a network, wherein the user conditions the workstation to enter surgical operation related data and to communication the entered data to the acquisition processor when the acquisition processor is acquiring said surgical operation related data.

9. A system according to claim 8 wherein the workstation comprises a display device for displaying the single display image, and a user input device allowing a user to interact with the single display image to enter the surgical operation related data.

10. A system according to claim 3 wherein the system further comprises a display device for displaying the single display image, and a user input device allowing a user to interact with the single display image to enter surgical operation related data.

11. A system according to claim 3, further comprising a database coupled to the acquisition processor, wherein the acquisition processor accesses the database when acquiring said surgical operation related data.

12. A system according to claim 3, wherein said single display image presents the user selectable data including clinical and non-clinical data of said particular patient and including at least one of, (a) medical assessment information of said particular patient provided by a physician, (b) a consent form associated with treatment of said particular patient and (c) a questionnaire associated with treatment of said particular patient.

13. A system according to claim 3, wherein said received data identifying said particular surgical operation is data identifying a particular set of orders for treatments and related services initiated for processing by a physician and associated with said particular surgical operation.

14. A system according to claim 13, wherein said acquisition processor automatically acquires said surgical operation related data in response to a physician initiating processing of said particular set of orders via a treatment order entry system.

15. A system according to claim 13, wherein said display processor terminates processing of said display image in response to cancellation of said particular set of orders via a treatment order entry system.

16. A system according to claim 3, further comprising an authorization processor for inhibiting said display processor from providing data representing said single display image in response to a determination, based on received user identification information, that a user is unauthorized to access said surgical operation related data of said particular patient.

17. A system according to claim 3, wherein said acquisition processor bidirectionally communicates with a plurality of different information sources in acquiring said surgical operation related data.

18. A system according to claim 3, further comprising a configuration processor enabling a user to determine information to be presented in said display image including at least one of, (a) said user selected time period and (b) said user selected items of surgical operation related data.

19. A system according to claim 3, wherein said display processor provides data representing an image window enabling a user to select said particular surgical operation of said particular patient from data representing a plurality of different operations performed on said particular patient.

20. A system according to claim 3, wherein said single display image identifies two or more of, (a) date and time of surgery, (b) participating surgeons and physicians and (c) place of surgery.

21. A system according to claim 3, wherein said single display image includes display attributes for at least one of, (a) highlighting the day of surgery and (b) highlighting the scheduled day of surgery.

22. A system according to claim 3, wherein said acquisition processor acquires said surgical operation related data in response to user manual data entry.

23. A system according to claim 3, wherein said user selected items of surgical operation related data comprise patient clinical medical parameters.

24. A user interface system for presenting medical information supporting a patient surgical procedure and related treatment, comprising:
an acquisition processor for automatically acquiring surgical operation related data including intra-operative data and at least one of, pre-operative data, and post-operative data associated with a particular surgical operation of a particular patient in response to received data identifying a particular set of orders for treatments and related services initiated for processing by a physician and associated with said particular surgical operation;
a display processor for providing data representing a single display image presenting a timeline together with user selected items of surgical operation related data acquired by said acquisition processor for a patient during a user selected time period; and
a command processor for initiating presentation of said single display image in response to user command.
